(19) United States
(12) Patent Application Publication
(10) Pub. No.: US 2006/0057065 A1
(43) Pub. Date: Mar. 16, 2006

(54) Composition and method to prevent and treat brain and spinal cord injuries

(76) Inventor: Yanming Wang, Malden, MA (US)

Correspondence Address: YANMING WANG, 203 SUMMER STREET, MALDEN, MA, 02148, US

(21) Appl. No.: 10/939,253

(22) Filed: Sep. 11, 2004

(51) Int. Cl. A01N 1/02; A61K 38/28; A61K 49/00; A61K 31/70

(52) U.S. Cl. 424/9.1; 514/3; 514/47; 514/23; 435/1.1

(57) ABSTRACT

The cerebrospinal fluid (CSF) contains low concentration of albumin and insulin because of the blood-CSF barrier. This is the major reason for cerebral edema and the resultant blood perfusion deficit when brain or spinal cord is injured. A composition and method for treating brain and spinal cord are provided. The composition includes magnesium, colloidal osmotic agent, insulin and ATP in artificial CSF. The method includes withdrawing a volume of cerebrospinal fluid from the subarachnoid space and infusing the invented composition.

### BACKGROUND OF THE INVENTION

1. Field of the Invention

This invention is related to a medical formulation for protecting the central nervous system and method of using the formulation. In particular, the invention relates to a neuroprotective composition and method using the composition to protect the brain and spinal cord or minimize lasting damage.

2. Background Information

Central nervous system (CNS) consisting of the brain and spinal cord is very vulnerable to injuries. Current search for a neuroprotective treatment based on various molecular mechanisms has yielded a disappointing result during clinical trial. One possible reason for these failures is because of the negligence of blood perfusion deficit following an initial injury. It has been known that after cardiac arrest and global ischemia, the brain suffers a “no-reflow” phenomenon. In the 1960s, Ames and coworkers produced global cerebral ischemia for 6 minutes in rabbits followed by carbon black ink infusion. They found that a large amount of the brain suffered from perfusion deficits. Similar to the “no-reflow’ phenomenon, post-ischemic or post-traumatic “hypoperfusion” has also been documented after spinal cord and brain injuries. The pharmaceutical intervention has seldom targeted this blood perfusion deficit. All CNS injuries such as hypoxia-ischemia, trauma, infection, poisoning etc. are invariably associated with cerebral edema which is a common pathway leading to cell death. It has been proposed that cerebral edema might play a major role for this blood perfusion deficit. Clinical prevention and treatment for cerebral edema include intravenous administration of osmotic agent, diuretic, coticosterroids etc, however, the efficacy is always temporary and limited.

Our scientific community is familiar with hydrocephalus and cytotoxic, vasogenic cerebral edema, yet the cerebrospinal fluid (CSF), the huge water and electrolytes resource, have seldom been linked with cerebral edema and the vulnerability of the brain and spinal cord to injuries. The existence of the CSF is a unique feature of the CNS. In an adult human, the CSF volume ranges from about 52 to 160 ml (mean 140 ml), occupying 10 percent of the intra-cranial and intraspinal volume. The average rate of CSF formation is about 21 to 22 ml/hr, or approximately 500 ml/day. The CSF as a whole is renewed four or five times daily. The choroid plexuses are the main sites of CSF formation. They consist of highly vascularized, “cauliflower-like” masses of pia mater tissue. Capillaries in the choroid plexus are highly specialized for their function. Unlike those in the rest of the cerebral vessels, their capillaries are fenestrated, non-continuous and have gaps between the capillary endothelial cells allowing the free-movement of small molecules. However, the adjacent choroidal epithelial cells are linked by tight junctions preventing most macromolecules from effectively passing into the CSF from the blood. The epithelium thus forms what is known as the blood-CSF barrier. The CSF is secreted by the epithelial cells of the choroid plexuses involving the transport of ions across the epithelium from blood to CSF. Being considered a part of the extracellular fluid, the CSF might be actively engaged in maintaining the chemical environment and metabolizing of the CNS. The composition of small molecular weight chemicals such as water, electrolytes, glucose, lactate is similar to that in plasma (CSF: Na⁺ 141 mEq/L, K⁺ 3.3 mEq/L, Mg⁺⁺ 2.4 mEq/L, Ca⁺⁺ 2.5 mEq/L, C⁻ 124 mEq/L; Glucose 61 mg/dl. Lactate 1.7 mEq/L. Plasma: Na⁺ 137 mEq/L, K⁺ 4.9 mEq/L, Mg⁺⁺ 1.64 mEq/L, Ca⁺⁺ 5.0 mEq/L, C⁻ 101 mEq/L, Glucose 92 mg/dl, Lactate 1.7 mEq/L). However, larger molecular chemicals are not permeable to enter the CSF. For examples, the CSF contains very low concentration of Albumin and insulin. The CSF formation is related to intracranial pressure (ICP). When the ICP is below about 70 mm H₂O, the CSF is not absorbed, and production increases. The CSF also serves as a kind of water jacket for the spinal cord and brain effectively making the weight of the brain and spinal cord 1/30th of its actual weight protecting them from potentially injurious blows to the spinal column and skull and acute changes in venous pressure. The CSF occupies the subarachnoid space, it has free access to neurons and surrounding glia cells through the Vichow-robin space which is also known as the perivascular spaces. Smaller blood vessels, which centripetally penetrate into the brain proper, are accompanied by an extension of the subarachnoid space that forms the Virchow-Robin space and is filled with the CSF.

Water makes up about 45-65% of total body weight in the human adult. It can enter cells through water channels. For examples, many water channels such as Aquapprin-1 (AQP1), Aquapprin-4 (AQP4), Aquapprin-5 (AQP5), Aquapprin-9 (AQP9) have been identified in CNS and believed to play an important role in the development of cerebral edema. The body fluid must be virtually in “gel form” which only allows small part of fluid to “free flow”. It mainly diffuses through the “gel”; that is, it moves molecule by molecule from one place to another by kinetic motion rather than by large numbers of molecules moving together. Excessive “free flow” fluid is one of the important basis of tissue edema. A colloidal osmotic agent is mainly for maintaining colloidal osmotic pressure (COP) and controlling body fluid in human body. Proteins from animal, plant and microbial source, gelatin, polypeptide can all function as colloidal osmotic agent. Albumin is the major colloidal osmotic agent in plasma, its water holding capacity is so large that it is estimated that one gram of albumin binds 18 ml of water. It measures about 6% in plasma (contributing 26 mm Hg COP), 2% in intercellular fluid and 3-4% in lymphatic fluid. The COP counteracts hydrostatic pressure preventing edema according to Starling's equation. The interstitial fluid and lymphatic fluid pressure at capillary level is believed to be very low or even negative. In CNS however, the CSF contains almost no COP because of the extremely low albumin concentration. The molecular weight of albumin is about 60,000 Daltons, it can not enter the CSF through choroid plexuses because of the brain-CSF barrier. The ICP which is the hydrostatic pressure of the CSF (normally ranged between 80-180 mm H₂O) is often elevated when brain and spinal cord are injured.

Insulin secretion from pancreatic β cells responds very precisely to small changes in glucose concentration in the physiologic range, hereby keeping blood glucose levels within the range of 70-150 mg/dL in normal individuals. It is “in charge” of facilitating glucose entry into cells. The CSF contains about two third of plasma glucose concentration (CSF: 61 mg/dl; Plasma: 92 mg/dl). However it contains about at most one fifteenth of plasma insulin concentration (CSF: 4 μU/ml; fasting plasma: 20-30 μU/ml). If considering fasting insulin in plasma the optimal biological effectiveness to normal blood glucose concentration, the insulin concentration in the CSF is at least three to four times lower than optimal. In addition, evidences have shown that CSF glucose concentration increases easily as plasma glucose concentration escalating, however there is no or very tiny increase of insulin in CSF as plasma insulin and glucose concentration escalating. Insulin is a small protein, with a molecular weight of about 6000 Daltons. It is not easy to enter the CSF through choroid plexuses because of the brain-CSF barrier. Plasma is main source of insulin supply for CNS tissue. Because the amount of insulin does not match the amount of glucose in the CSF, there is a relative insufficient amount of insulin to CNS. The insulin shortage will become apparent when the CNS tissue is damaged and followed by secondary blood perfusion deficit. The CNS is a surprisingly active tissue in terms of energy metabolism. Under aerobic condition, cerebral energy is mainly provided by glucose and oxygen through oxidative phosphorylation. Large amounts of energy are required to maintain the signaling activities of CNS cells. It is estimated that 49% of the oxidative energy and 58% of the glycolytic energy is consumed for Na⁺ transportation in neuronal tissue. The Na⁺ concentration is much higher in extracellular than that in intracellular. Since Na⁺ attracts more water molecules and is relatively not permeable to cell membrane, it becomes the only major force to hold water in CSF. Normally only small amount of Na⁺ influx is allowed through sodium channels because of the membrane depolarization and the excessive Na⁺ is quickly transferred out by Na K-ATPase. When the energy supply is compromised, excessive Na⁺ can not be pumped out of the cell leading to Na⁺ accumulation. It has been well documented that Na⁺ together with water are significantly increased in edematous tissue. As a defendant strategy, glucose metabolism switches from oxidative metabolism to anaerobic glycolysis (glucose to lactate) during ischemia-hypoxia. Glycolysis itself does not require oxygen and can proceed aerobically or anaerobically. Although glycolytic pathway is not efficient to provide energy compared with oxidative phosphorylation, it becomes crucial for the maintenance of neuronal activity and survival in cerebral ischemic condition. The increased ability to perform glycolysis following cerebral ischemic challenge is due to coordinated upregulation of the anzymes of glycolysis. Insulin activates glycolysis probably through activating phosphofructoskinase-2 (PFK-2). Mounting evidences have repeatedly proven that insulin can yield protection for ischemic cerebral tissue. It is not feasible to significantly increase plasma insulin level for a long period. Secondary blood perfusion deficit causes insufficient insulin supply to the damaged CNS tissue and unbalanced coupling of glucose and insulin in CSF. In rats, high concentration of insulin (2500 μU/ml) administered through third ventricle at rate of 5 μU/min does not result in significant plasma glucose reduction. However direct administering insulin to subarachnoid space for protecting CNS tissue have not been reported.

Glucose is the major energy source for CNS. Severe hypoglycemia cause coma and result in neuronal death. However, clinical and experimental studies have proven that hyperglycemia also exacerbate neuronal damage. Insulin can ameliorate hyperglycemia induced neuronal damage. The entry of glucose into cells must be facilitated by insulin. Therefore, although glucose is a fundamental factor in protecting CNS tissue, glucose and insulin must be in appropriate proportion.

The brain and spinal cord are submerged in the CSF which provides endless water and Na⁺ but low COP and insufficient insulin. We propose that these unique physiological characters of the CSF and anatomic features of the CNS place the brain and spinal cord in a very delicate edema prone position and might be the reasons why the brain and spinal cord are more vulnerable than other organs.

Magnesium (Mg²⁺) is the second highest electrolyte intracellularly (58 mEq/L). ATP (Adenosine 5′-triphosphate) is always present as a magnesium: ATP complex. Mg²⁺ basically provides stability to ATP. At least more than 260 to 300 enzymes have been found to require Mg²⁺ for activation. Best known among these are the enzymes involved in phosphorylations and dephosphorylations: AT-Pases, phosphatases, and kinases for glycolytic pathway and krebs cycles. At the level of the cell membrane Mg²⁺ is needed for cytoskeletal integrity, the insertion of protein into membranes, the maintenance of bilayer fluidity, binding of intracellular messengers to the membrane, regulation of intracellular Ca²⁺ release by inositol triphosphate etc. Mg²⁺ also affects the activities of pumps and channels regulating ion traffic across the cell membrane. The potential changes in tissue Mg²⁺ might also affect the tissue ATP levels. In tissue culture and animal models elevated Mg²⁺ concentration has been repeatedly proven to protect cells. The concentration of ATP inside cells is high, whereas the concentration outside cells is very low. Harkness and coworkers showed that the ATP concentrations is about 1 to 20 μmol/l in plasma, however in CSF, ATP could not be detected, and it was estimated to be about less than 0.05 μmol/l. Mufioz and coworkers detected that the ATP concentration in CSF is about 16 nM/l. Exogenous ATP provides direct energy to the damaged tissue. Sakama and coworkers showed that continuous application of ATP (100 μM) significantly increased axonal transport of membrane-bound organelles in anterograde and retrograde directions in cultured neurons. Uridine 5′-triphosphate produced an effect similar to ATP. Mg-ATP has been used clinically to protect hepatic and other cells after hypoxia-ischemia.

Acidosis is a universal response of tissue to ischemia. In the brain, severe acidosis has been linked to worsening of cerebral infarction. Recent evidence however suggests that mild extracellular acidosis protects the brain probably through preventing activation of NMDA receptors and inhibition of Na⁺/H⁺ exchange. It has been reported mild acidosis provide cell protection down to pH 6.2. The acidosis that accompanies ischemia is an important endogenous protective mechanism. Correction of acidosis seems to trigger the injury. It has also been speculated that mild acidosis might stimulate anaerobic glycolysis that might supplement NADH oxidation and ATP yields.

Recombinant tissue plasminogen activator (rt-PA), a thrombolytic agent, has been shown to be effective if used within 3 hours after the onset of the stroke.

U.S. Pat. No. 6,500,809 to Frazer Glenn discloses a hyperoncotic artificial cerebrospinal fluid and method of treating neural tissue edema. A series of patents, U.S. Pat. Nos. 4,981,691, 4,758,431, 4,445,887, 4,445,500, and 4,393,863 to Osterholm disclose an oxygenated fluorocarbon solution for treatment of hypoxic-ischemic neurological tissue.

### SUMMARY OF THE INVENTION

The CNS is very vulnerable to injuries. All CNS injuries are associated with secondary blood perfusion deficit induced by cerebral edema which is a common pathological process. All current clinical measures for prevention and treatment of CNS injuries induced edema only provide temporary and limited effect. Current search for a neuroprotective agent based on other molecular mechanisms has yielded a disappointing result during clinical trial.

Our hypothesis is as follow: the CSF has very low COP and insufficient amount of insulin because of the brain-CSF barrier. It is readily available to provide endless source of “free flow” water and Na⁺ to bath and exert pressure to the CNS tissue. When the brain or spinal cord is injured by an initiating insult such as ischemia or trauma, ATP production is reduced. This leads to massive Na⁺ and water molecules influx across membrane from the CSF resulting in rapid development of cell edema and eventually death. While excessive Na⁺ and water molecules inside the cell body is toxic, swelling of the cerebral tissue makes the Virchow-Robin space smaller and may even cause it to collapse, thereby compressing the small blood vessels and resulting in obstruction of the blood flow, such as a “hypoperfusion” or even “no-reflow” phenomenon, which prolongs the original ischemic duration, blocks collateral circulation and induces a feedback loop. Since plasma is the main source of insulin for CNS tissue, this secondary blood perfusion deficit amplifies the effect of insulin shortage in the CSF impairing intake of glucose and glycolysis. These cascade events result in irreversible cell death, tissue necrosis and liquefaction, finally leading to neurological deficits and even brain death. As the hydrostatic pressure of the CSF, the ICP promotes cerebral edema.

Although the existence of the CSF causes vulnerability of the CNS, it also provides an opportunity for treatment. Increasing the COP of the CSF, adding more glucose, ATP and insulin in the CSF and lowering ICP will reduce the cerebral edema herein increasing the cerebral flow and protecting the brain and spinal cord tissue. Elevated Mg²⁺ concentration and mild acidosis environment in CSF will enhance glycolytic capacity increasing the tolerant ability of cerebral tissue to ischemic injury.

This invention provides composition which contains a mixture of a COP agent, insulin, glucose, ATP and increased Mg²⁺ concentration in artificial CSF. This invention also provides a method of using the composition to protect brain and spinal cord. The composition and method according to this invention may be used to treat cerebral edema induced by neurological disorders, such as stroke, hypoxia-ischemia, hemorrhage, head and spinal cord trauma, multiple sclerosis, seizure, infection and poisoning etc. The composition and method may be useful for preventing cerebral edema during open-heart surgery, neurosurgery, aortic surgery, shock, or other procedures where blood flow to the CNS needs interrupting. Neurosurgical procedures routinely require that application of fairly copious amounts of various liquids to replace lost CSF and to irrigate and protect the CNS. Neurosurgeons are stilling use saline to irrigant during the surgical procedures. The compositions and method may be also useful to replace saline during neurosurgical procedures.

I have found the composition I have used is effective when it is applied to the subarachnoid spaces after the CSF has been removed completely or partially from the subarachnoid spaces. This method is effective to treat injured CNS tissue or to protect it from continuing damage after injury. To treat or prevent the CNS injuries, the composition will be injected into subarachnoid space around the injured CNS tissue. Optionally, the composition can also be administered continuously through one infusing catheter and one draining catheter positioned in subarachnoid spaces between the cerebral tissue where protection is needed. The high concentration albumin or gelatin increases COP of the composition limiting “free flow” water. The presence of insulin and glucose, the elevated Mg²⁺ concentration and the mild acidosis in the composition increase glycolytic capacity to yield more ATP production. Exogenous ATP can provide additional energy to the damaged tissue. Elimination of cerebral edema prevents the onset of the “no-reflow” phenomenon or “hypoperfusion”, making CNS tissue resistant to injuries, and lengthening the therapeutic window for all other therapies.

There are many advantages to the composition and method I have discovered.
1. The compositions includes a combination of high concentration COP agent, glucose, insulin, ATP and increased Mg²⁺ concentration in artificial CSF. Since all components have been uses clinically and proven to be safe, the composition and method provide an effective and simple approach to protect brain and spinal cord.
2. It improves the efficacy of existing treatments for stroke, head trauma, and other invasive procedures. Administering an effective composition through subarachnoid spaces after removing the CSF according to this invention will increase the therapeutic window for any other treatment, including thrombolytic agents. For example, tPA, the only FDA approved medication for stroke, a thrombolytic agent targeting blood clot, is now only approved for use within 3 hours after onset of ischemia. When used in combination with the instant composition and method, the therapeutic window for all known treatments now used for supporting CNS tissue will be much longer. Another example, if this composition is used before, during and after brain and spinal cord surgery, a quicker and better recovery result can be achieved.
3. This invention also can reduce the cost of the treatment. Albumin is expensive, for example, hospital units have shown that albumin accounts for 10 to 30% of pharmacy budgets. This invention provides many effective alternative COP agents that is less expensive, such as gelatin.
4. This invention, if combined with other known techniques such as controlled hypothermia, may significantly increase the length of time a patient can tolerate cerebral ischemia. A patient treated according to this invention may help survive invasive procedures performed on any part of the CNS, including brain stem. Additionally, procedures that require interruption of the blood flow, such as heart surgery, repair of aortic aneurysm, or any other surgery where systemic blood circulation is interrupted can be performed with increased safety. The compositions and method I have invented extend the therapeutic window for successfully resuscitating cardiac arrest from mere minutes to hours.

### DETAILED DESCRIPTION OF AN ILLUSTRATIVE EMBODIMENT

As we discussed in the above, all CNS injuries are associated with secondary blood perfusion deficit induced by cerebral edema which is a common pathological process. It has been well documented that Na⁺ and water are significantly increased in edematous tissue. In CNS, edema formation is even quicker and stronger because the CSF provides large amount of Na⁺ and water in addition to that from blood. The CSF contains large amount of water and Na⁺. However, owing to the blood-CSF barrier, it contains slightly low concentration of glucose and extremely low albumin and tiny amount of insulin. The CSF occupying the subarachnoid space has free access to neurons and surrounding glia cells through the Vichow-robin space. It is readily available to bath and exert pressure to the CNS tissue. When the brain or spinal cord is injured by an initiating insult such as ischemia or trauma, besides Na⁺ and water molecules from plasma, energy failure results in Na⁺ and water massive influx from the CSF across membrane. Na⁺ and water accumulation leads to rapid development of cell edema. Swelling of the cerebral tissue makes the Virchow-Robin space smaller and may even cause it to collapse, thereby compressing the small blood vessels and resulting in an obstruction of the blood flow, such as a “hypoperfusion” or even “no-reflow” phenomenon, which prolongs the original ischemic duration, blocks collateral circulation and induces a feedback loop. As the hydrostatic pressure of the CSF, the ICP can deduct the force of colloidal osmotic pressure promoting edema. Since plasma is is the main source of insulin for CNS tissue, this secondary blood perfusion deficit will worsen the effect of insulin shortage in the CSF impairing intake of glucose and glycolysis.

Based on human CSF, I have invented a composition comprising a mixture of COP agent, insulin, glucose, ATP and elevated Mg²⁺ concentration in artificial CSF to prevent and treat brain and spinal cord injuries. There are many COP agents can be chosen, such as, proteins (the protein can be selected from any source, such as animal, vegetable, or microbial, without limitation, the protein can also be modified to increase the ability to absorb water), collagen, fibrin, gelatin. GELOFUSINE® (containing 4-14% gelatin) from Millpledge Ltd can also be chosen. HAEMACCEL® 3.5% colloidal intravenous infusion solution containing gelatin polypeptides being used in clinic in South Africa can also be chosen. Heat shock protein and thrombin can also be chosen. However albumin and gelatin are preferred. The concentration of COP agent should be sufficient to create COP between 1 to 200 mm Hg. It is preferred that COP agent can create a pressure of 20-40 mm Hg. 8% albumin (8 gram in 100 ml solution) creating about 33 mm Hg COP is most preferred. 3-4% gelatin creating about 20-40 mm Hg COP is most preferred too. Glucose concentration should be in a range from 1 to 480 mg/dl. The preferred glucose concentration is between 21 to 240 mg/dl. The most preferred glucose concentration is between 21 to 120 mg/dl. The insulin concentration should be in a range from 0.01 to 1000 μU/ml. The preferred insulin concentration is between 5 to 60 μU/ml. Fructose-2,6-biphosphate is the most potent stimulator of key enzyme of glycolysis, therefore it can be chosen to replace insulin. All growth factors have insulin-like effect and can be chosen to replace insulin. Growth hormones and growth hormone releasing factor have insulin-like effect, can also be chosen to replace insulin. For examples, insulin-like growth factors, nerve growth factor, brain derived neurotrophic factor, neurotrophin, fibroblast growth factor and glial cell line derived neurotrophic factor, erythroproietin, growth hormone, growth hormone releasing factor etc may be used to replace insulin or may be used in combination with insulin. The ATP concentration should be in a range from 16 nM to 5 mM. The preferred ATP concentration is between 0.001 to 5 mM. The most preferred ATP concentration is between 0.001 to 0.05 mM. Other high energy compound such as Uridine 5′-triphosphate can be used to replace ATP. The artificial CSF used for research usually contains minimums Mg²⁺ of 0.9 meq/L. The elevated Mg²⁺ concentration should be in a range from 0.91 to 10 meq/L. The most preferred Mg²⁺ concentration is between 2.51 to 5.0 meq/L. Normal blood pH value is about 7.35 to 7.45. The pH value of the composition should be in a range between 6.2 to 7.35. The pH value between 6.8-7.0 is preferred. The pH value may be adjusted by phosphate buffer, hydrochloric acid or by bicarbonate. Table 1 shows the components and concentration range of the composition.

TABLE 1

| Component | Concentration range | |
|---|---|---|
| Artificial CSF | | |
| Na | 120-155 | meq/L |
| K | 0.1-5.0 | meq/L |
| Ca | 0.1-3.0 | meq/L |
| P | 0.1-10 | meq/L |
| Cl | 120-155 | meq/L |
| Mg | 2.51-5 | meq/L |
| Glucose | 1-240 | milligram/dl |
| Albumin | 0.1-20 | gram/dl |
| Insulin | 5 to 60 | μU/ml |
| ATP | 0.001 to 0.05 | mM |

(Use sterile water for dilution, pH is adjusted between 6.8-7.3, osmolality adjusted between 280-310 mOsm/L, COP between 20-40 mm Hg)

Optionally the albumin in Table 1 may be replaced by gelatin 0.1-10 gram/dl.

The mixture of albumin (or gelatin), insulin, ATP, glucose, and Mg²⁺ and mild acidosis have synergic neuroprotective effects. However each individual component dissolving in artificial CSF is also effective and can be used alone.

To make the composition, albumin (or gelatin), insulin, ATP and elevated Mg²⁺ concentration in artificial CSF may be manufactured in a ready to use condition. Optionally, artificial CSF with elevated Mg²⁺ concentration may be manufactured in one container, the mixture of albumin (or gelatin), insulin and ATP may be assembled in another container. Since albumin (or gelatin), insulin and ATP are delicate substances, it would be convenient and advantageous to keep their mixture in a cool place and dissolving them in artificial CSF just before use. For example, mixture of albumin (or gelatin), insulin and ATP may be assembled in different quantities in small mapules that is ready for being dissolved in 10 ml, 20 ml, 50 ml, 100 ml and 500 ml of the artificial CSF.

The composition can also be added any of other nutrients such as, Vitamins (such as, D-Calcium Pantothenate, Choline, Folic acid, i-Inositol, Niacinamide, Pyridoxal, Riboflavin, Thiamine, Vitamin B₁₂ etc.), Amino acids (such as, L-Alanine, L-Arginine, L-Asparagine, L-Cysteine, L-glutamine, L-glutamate, Glycine, L-Histidine, L-Isoleucine, L-leucine, L-lysine, L-methionine, L-Phenylalanine, L-proline, L-serine, L-threonine, L-tryptophan, L-tyrosine, L-valine etc.), phospholipids, Cholesterol, fat, fatty acid, D,-L-alpha-tocopherol, antioxidant etc. The composition can also be added any of oxygen carriers such as bis-perfluorobutyl ethylene and oxygenated before use. The composition can also be added any of intermediates of glycolysis (such as fructose-1,6-biphophate, glyceraldehyde-3-phosphate, 1,3 bisphosphoglycerate, 3-phosphoglycerate, 2-phosphoglycerateare, phosphoenolpyruvate, pyruvate, lactate etc.), enzymes for glycolysis (such as hexokinase, phosphoglucose isomerase, phosphofructokinase, aldolase, triosephosphate isomerase, glyceraldehydes 3-phosphate dehydrogenase, phosphoglygerate kinase, pyruvate kinase etc.), ketone bodies (such as acetoacetate, β-hydroxybatyrate) and intermediates of krebs cycle.

The compositions herein may also be advantageously combined with any of the agents used to treat stroke or other neurological deficiencies based on other mechanisms including: calcium channel blockers such as Nimodipine, and Flunarizine; calcium chelators, such as DP-b99; potassium channel blockers; Free radical scavengers—Antioxidants such as Ebselen, porphyrin catalytic antioxidant manganese (III) meso-tetrakis (N-ethylpyridinium-2-yl) porphyrin, (MnTE-2-PyP (5+)), disodium 4-[(tert-butylimino) methyl] benzene-1,3-disulfonate N-oxide (NXY-059), N:-t-butyl-phenylnitrone or Tirilazad; GABA agonists including Clomethiazole; GABA receptor antagonists, glutamate antagonists, including AMPA antagonists such as GYKI 52466, NBQX, YM90K, YN872, ZK-200775 MPQX, Kainate antagonist SYM 2081, NMDA antagonists, such as CGS 19755 (Selfotel); NMDA channel blockers including Aptiganel (Cerestat), CP-101,606, Dextrorphan, destromethorphan, magnesium, metamine, MK-801, NPS 1506, and Remacemide; Glycine site antagonists including ACEA 1021, and GV 150026; polyamine site antagonists such as Eliprodil, and Ifenprodil; and adenosine receptor antagonists; Growth factors such as Fibroblast Growth Factor, brain derived neurotrophic factor, insulin like growth factor, neurotrophin. Nitric oxide inhibitors including Lubeluzole, opiod antagonists, such as Naloxone, Nalmefenem, Phosphatidylcholine precursor, Citicoline (CDP-coline); Serotonin agonists including Bay x 3072; Sodium channel blockers such as Fosphenyloin, Lubeluzole, and 619C89; Potassium channel openers such as BMS-204352; anti-inflamatory agents; protein kinase inhibitors and other active agents that provide energy to cells, such as co-enzyme A, co-enzyme Q, or cytochrome C. Similarly, agents known to reduce cellular demand for energy, such as phenyloin, barbital, or lithium may also be added. These agents may be added into this invented composition or may be administered orally or intravenously in combination with this invented composition and method.

To use the compositions to treat or prevent localized brain or spinal cord injuries, such as stroke, head or spinal cord trauma, one catheter may be placed through a puncture in the lumbar theca or cistema magna or subarachnoid spaces in direct affected area. Optionally, for maximum CNS tissue protection, another catheter may be placed into the subarachnoid spaces around injured cerebral tissue, additional catheters may be inserted into the lateral cerebral ventricles as general CNS protection is needed. The CSF is withdrawn from one or all of these locations to remove CSF to cut off the major water and Na⁺ supply to cerebral tissue (usually 5-200 ml) and to reduce the ICP. By removing the CSF, the ICP can be reduced even to 0 mm H₂O if necessary. After removal of the CSF, slowly injecting invented composition to the affected area of the CNS tissue. The injected composition is approximately equal or less to the amount of CSF removed, the ICP will be reduced or at least not be increased. Optionally, if whole CNS needs to be protected such as cardiac arrest or during the cardiac surgery, severe head trauma, stroke etc., the CSF should be removed as completely as possible, the treatment composition can be infused continuously from an infusing catheter to a draining catheter in subarachnoid spaces. The infusing rate of the composition can be from 0.001-100 ml/min. The treatment procedure can be repeated. Optionally, the composition may be cooled between 4 to 37° C. before use. Alternatively, to treat or prevent brain or spinal cord injury, Patient's own CSF may be used to replace artificial CSF in our composition. Usually 5-160 ml of the patient's own CSF can be obtained as a solvent to dissolve the mixture of albumin (or gelatin), insulin and ATP. Elliot B solution is an artificial CSF that has been approved as a solvent since 1996 in USA. Elliot B solution may also be used to replace artificial CSF in our composition. The composition can be removed from the subarachnoid space if necessary following patient's recovery.

Meanwhile, combined with this invention, administering agent to suppress production of CSF can be advantageous. There are many known agents inhibiting production of CSF. These agents include all diuretics, such as furosemide (20-200 mg every 4-6 hours), and acetazolamide (0.25-2 g every 4-12 hours). These agents if administered intravenously or orally may enhance the efficacy of invented composition and treatment method.

The composition and method can be combined with and enhance the efficiency of thrombolytic agents such as: recombinant tissue plasminogen activator (rtpA), streptokinase, and tenecteplase in dissolving thrombosis in management of stroke.

### EXAMPLE ONE

Making of a Composition for Protecting CNS Tissue

Artificial CSF used in this example is made according to table 2.

TABLE 2

| Component | Amount | |
|---|---|---|
| NaCl | 8.182 | gram |
| KCl | 0.224 | gram |
| CaCl₂.2H₂O | 0.206 | gram |
| Na₂HPO₄ | 0.113 | gram |
| NaH₂PO₄ | 0.023 | gram |
| MgSO₄ | 0.361 | gram |
| Glucose | 0.6 | gram |

Sterile water for dilution to 1000 ml

Mixture of Albumin, Insulin and ATP used in this example is made according to table 3.

TABLE 3

| | | |
|---|---|---|
| Albumin | 80 | gram |
| Insulin | 30 | International Units |
| ATP | 5.5 | milligram |

Mix these substances in one container

To make the composition, dissolve the mixture of Albumin, Insulin and ATP in artificial CSF. Final pH of the composition is adjusted between 6.8 to 7.0.

### EXAMPLE TWO

Making of a Composition for Protecting CNS Tissue

Artificial CSF used in this example is made according to table 2 in example one.

Mixture of Gelatin, Insulin and ATP used in this example is made according to table 4.

TABLE 4

| | | |
|---|---|---|
| Gelatin | 30 | gram |
| Insulin | 30 | International Units |
| ATP | 5.5 | milligram |

Mix these substances in one container

To make the composition, dissolve the mixture of Gelatin, Insulin and ATP in artificial CSF. Final pH of the composition is adjusted between 6.8 to 7.0.

### EXAMPLE THREE

Treatment for Brain Ischemia

The focal cerebral ischemia was induced in 12 rats weighing between 250-300 gram. Group one: control (6 rats). Group two: treatment with the composition made according to example one (6 rats). Group three: treatment with the composition made according to example two (6 rats). Ketamine/xylazine 30 mg/kg ip was given for anesthesia. A silicone catheter (0.025 OD, 0.012 ID inch) was surgically implanted in the cisterna magna as a draining route. A hole of 3 mm in diameter was drilled on the left side of skull (3 mm lateral to midline and 3 mm in front of the bregma), dura was punctured, an infusing silicone catheter (0.025 OD, 0.012 ID inch) was placed and fixed with glue in the hole into the subarachnoid spaces on the surface of the forebrain.

Focal cerebral ischemia was produced by middle cerebral artery occlusion. A midline incision on the neck was made. The left common carotid artery, the external carotid artery (ECA) and the internal carotid artery (ICA) were exposed. The ECA was ligated and severed. A 3.0 nylon suture was advanced from the ECA to ICA to block the origin of left middle cerebral artery. The nylon suture was left in place for 3 hours to produce focal cerebral ischemia on left hemisphere supplied by middle cerebral artery. Then the nylon suture was removed to resume blood supply to the ischemia challenged brain for 21 hours.

For group one, rats received artificial CSF at 15 min after ischemia (Table 4).

TABLE 4

| Component | Amount | |
|---|---|---|
| NaCl | 8.66 | gram |
| KCl | 0.224 | gram |
| CaCl₂.2H₂O | 0.206 | gram |
| Na₂HPO₄ | 0.113 | gram |
| NaH₂PO₄ | 0.023 | gram |
| MgSO₄ | 0.096 | gram |

Sterile water for dilution to 1000 ml, pH adjusted to 6.8-7.4

For group two, at 15 minutes after ischemia, the CSF was removed as completely as possible (usually 0.01-0.02 ml CSF could be withdrawn). After the CSF removal, 3 ml of the composition made according to example one was continuously infused from the catheter on the left forebrain and was drained out from the catheter in cisterna magna. The infusion lasted for 3 hours at a rate of 1 ml/hour.

For group three, at 15 minutes after ischemia, the CSF was removed as completely as possible (usually 0.01-0.02 ml CSF could be withdrawn). After the CSF removal, 3 ml of the composition made according to example two was continuously infused from the catheter on the left forebrain and was drained out from the catheter in cisterna magna. The infusion lasted for 3 hours at a rate of 1 ml/hour.

Behavioral deficit study: At 21 hours after cerebral ischemia, all rats were evaluated for behavioral deficit. A score of 0-4 was used to assess the motor and behavioral changes. Score 0: No apparent deficits. Score 1: Contralateral forelimb flexion. Score 2: Decreased grip of the contralateral forelimb while tail pulled. Score 3: Spontaneous movement in all directions; contralateral circling only if pulled by tail. Score 4: Spontaneous contralateral circling.

2,3,5-triphenyltetrazolium chloride (TTC) staining: After behavioral test, all rats were euthanized. Brains were taken out. TTC staining was used to distinguish the viable tissue and necrotic tissue. Sections of 1 mm in thickness were cut and stained with 2% TTC in phosphate buffer at 37° C. for 10 minutes. The sections were fixed in 10% formalin. Percentage of infarct volumes were calculated and analyzed with a computer.

Results: in group one, the average score was 3.67±0.52, the average infarct volume was 37±1.79%. In group two, the average score was 0.83±0.75, the average infarct volume was 2.83±0.75%. In group three, the average score was 0.67±0.52, the average infarct volume was 2.67±0.82%. It was concluded that the composition made according to example one and composition made according to example two significantly protected ischemia challenged brain (P<0.05).

While my above description contains many specifics, these should not be construed as limitations on the scope of the invention, but rather as illustrative examples.

1. A composition for protecting brain and spinal cord of a mammal comprising a mixture of at least one component selected from the group consisting of colloidal osmotic agent, insulin and ATP in an artificial cerebrospinal fluid.

2. A composition for protecting brain and spinal cord of a mammal comprising essentially a mixture of at least one component selected from the group consisting of colloidal osmotic agent, insulin and ATP in an artificial cerebrospinal fluid.

3. A composition for protecting brain and spinal cord of a mammal, as claimed in claim 1, wherein said artificial cerebrospinal fluid comprises of: Na 120-155 meq/L, K 0.1-5.0 meq/L, Ca 0.1-3.0 meq/L, P 0.1-10 meq/L, Cl 120-155 meq/L, Mg 2.1-5 meq/L, Glucose 1-240 mg/dl and water.

4. A composition for protecting brain and spinal cord of a mammal, as claimed in claim 1, wherein said colloidal osmotic agent is albumin.

5. A composition for protecting brain and spinal cord of a mammal, as claimed in claim 4, wherein said albumin is present in a concentration of about 0.1-20 gram per 100 ml.

6. A composition for protecting brain and spinal cord of a mammal, as claimed in claim 1, wherein said colloidal osmotic agent is gelatin.

7. A composition for protecting brain and spinal cord of a mammal, as claimed in claim 6, wherein said gelatin is present in a concentration of about 0.1-20 gram per 100 ml.

8. A composition for protecting brain and spinal cord of a mammal, as claimed in claim 1, wherein said insulin is present in a concentration of about 0.01 to 1000 μU/ml.

9. A composition for protecting brain and spinal cord of a mammal, as claimed in claim 1, wherein said ATP is present in a concentration of about 0.001 mM to 5 mM.

10. A composition for protecting brain and spinal cord of a mammal according to claim 1, further comprises at least one component selected from the group consisting of: Vitamins (such as, D-Calcium Pantothenate, Choline, Folic acid, i-Inositol, Niacinamide, Pyridoxal, Riboflavin, Thiamine, Vitamin B₁₂ etc.), Amino acids (such as, L-Alanine, L-Arginine, L-Asparagine, L-Cysteine, L-glutamine, L-glutamate, Glycine, L-Histidine, L-Isoleucine, L-leucine, L-lysine, L-methionine, L-Phenylalanine, L-proline, L-serine, L-threonine, L-tryptophan, L-tyrosine, L-valine etc.), phospholipids, Cholesterol, fat, fatty acid, D,-L-alpha-tocopherol, oxygen carriers (such as bis-perfluorobutyl ethylene and oxygenated before use), intermediates of glycolysis (such as fructose-1,6-biphophate, glyceraldehyde-3-phosphate, 1,3 bisphosphoglycerate, 3-phosphoglycerate, 2-phosphoglycerateare, phosphoenolpyruvate, pyruvate, lactate), enzymes for glycolysis (such as hexokinase, phosphoglucose isomerase, phosphofructokinase, aldolase, triosephosphate isomerase, glyceraldehydes 3-phosphate dehydrogenase, phosphoglygerate kinase, pyruvate kinase etc.), ketone bodies (acetoacetate, β-hydroxybatyrate), intermediates of krebs cycle, calcium channel blockers, calcium chelators, Sodium channel blockers, potassium channel blockers, potassium channel openers, free radical scavengers—Antioxidants, GABA agonists, GABA receptor antagonists, polyamine site antagonists, Glycine site antagonists, protein kinase inhibitors, Serotonin agonists, Nitric oxide inhibitors, opiod antagonists, glutamate antagonists, AMPA antagonists, adenosine receptor antagonists, Kainate antagonist, NMDA antagonists (such as CGS 19755, Nimodipine, DP-b99 and Flunarizine, Aptiganel, CP-101,606, Dextrorphan, destromethorphan, metamine, MK-801, NPS 1506, GYKI 52466, NBQX, YM90K, YN872, ZK-200775, MPQX, SYM 2081, Bay x 3072, Remacemide, ACEA 1021, GV 150026, Clomethiazole, Eliprodil, Ifenprodil, Lubeluzole, Naloxone, Nalmefenem, Citicoline, Fosphenyloin, Lubeluzole, 619C89, BMS-204352), Growth factors (such as nerve growth factor, Fibroblast Growth Factor, brain derived neurotrophic factor, insulin like growth factor, neurotrophin, erythroproietin, growth hormones, growth hormone releasing factor), and other active agents that provide energy to cells (such as co-enzyme A, co-enzyme Q, or cytochrome C), agents known to reduce cellular demand for energy (such as phenyloin, barbital, or lithium).

11. A composition for protecting brain and spinal cord of a mammal according to claim 1, has pH value of about 6.8 to 7.3

12. A method for protecting brain and spinal cord in a mammal, comprising the steps of:
a). Withdrawing a volume of cerebrospinal fluid from the subarachnoid space,
b). Infusing a said composition in an effective amount according to claim 1 into said subarachnoid space around brain and spinal cord where protection is needed.

13. A method for protecting brain and spinal cord in a mammal according to claim 12, comprising added step of: Administering agent to lower plasma Na⁺ and agent to reduce the CSF production.

14. A method for protecting brain and spinal cord in a mammal according to claim 13, wherein said agent to lower plasma Na⁺ and agent to reduce the CSF production are Furosemide and acetazolamide.

15. A method for protecting brain and spinal cord in a mammal according to claim 13, comprising further added step of administering an effective amount of agent selected from the group consisting of: calcium channel blockers, calcium chelators, Sodium channel blockers, potassium channel blockers, potassium channel openers, free radical scavengers—Antioxidants, GABA agonists, GABA receptor antagonists, polyamine site antagonists, Glycine site antagonists, protein kinase inhibitors, Serotonin agonists, Nitric oxide inhibitors, opiod antagonists, glutamate antagonists, AMPA antagonists, adenosine receptor antagonists, Kainate antagonist, NMDA antagonists (such as CGS 19755, Nimodipine, DP-b99 and Flunarizine, Aptiganel, CP-101,606, Dextrorphan, destromethorphan, metamine, MK-801, NPS 1506, GYKI 52466, NBQX, YM90K, YN872, ZK-200775, MPQX, SYM 2081, Bay x 3072, Remacemide, ACEA 1021, GV 150026, Clomethiazole, Eliprodil, Ifenprodil, Lubeluzole, Naloxone, Nalmefenem, Citicoline, Fosphenyloin, Lubeluzole, 619C89, BMS-204352), Growth factors (such as nerve growth factor, Fibroblast Growth Factor, brain derived neurotrophic factor, insulin like growth factor, neurotrophin, erythroproietin, growth hormones, growth hormone releasing factor), and other active agents that provide energy to cells (such as co-enzyme A, co-enzyme Q, or cytochrome C), agents known to reduce cellular demand for energy (such as phenyloin, barbital, or lithium).

16. A method for treating ischemic stroke in a mammal requiring such treatment according to claim 13 comprising added step of administering a thrombolytic agent to said mammal in an amount effective to restore blood flow to central nervous system tissue.

17. A method for treating ischemic stroke in a mammal requiring such treatment according to claim 16 wherein said thrombolytic agent is recombinant tissue plasminogen activator (rt-PA).

18. A composition for protecting brain and spinal cord of a mammal comprising a mixture of at least one component selected from the group consisting of colloidal osmotic agent, insulin and ATP in an artificial cerebrospinal fluid, wherein said artificial cerebrospinal fluid has Mg²⁺ concentration between 2.51 to 5.0 meq/L.

19. A composition for protecting brain and spinal cord of a mammal comprising a mixture of at least one component selected from the group consisting of insulin and ATP in an artificial cerebrospinal fluid, wherein said artificial cerebrospinal fluid contains colloidal osmotic agent.
